(19) United States
(12) Patent Application Publication
(10) Pub. No.: US 2006/0133575 A1
(43) Pub. Date: Jun. 22, 2006

(54) X-ray needle apparatus and method for radiation treatment

(75) Inventors: George Gutman, Birmingham, MI (US); Emil Strumban, Oak Park, MI (US)

Correspondence Address: BROOKS KUSHMAN P.C., 1000 TOWN CENTER, TWENTY-SECOND FLOOR, SOUTHFIELD, MI, 48075, US

(73) Assignee: Advanced X-Ray Technology, Inc., Birmingham, MI (US)

(21) Appl. No.: 11/190,424

(22) Filed: Jul. 27, 2005

Related U.S. Application Data
(60) Provisional application No. 60/638,016, filed on Dec. 21, 2004.

(51) Int. Cl. H05G 2/00; G21G 4/00; H01J 35/00; A61N 5/10

(52) U.S. Cl. 378/119; 378/65

(57) ABSTRACT

The invention is directed to an x-ray device and method for radiation treatment comprising an x-ray source 1, a collimator 4 incorporating conditional optics, such as a capillary lens 3 for directing and focusing the x-ray radiation, and implantable needles. One or more capillary semilenses 16, 17 are positioned along the optical axis of the x-ray beam allow to form a movable focus by changing the distance between the semilenses. The input end of the collimator 4 is optically and mechanically conjugated with the x-ray source 1. The output end of the collimator is optically and mechanically conjugated with an originating end of the needle 5. At its output end is a transparent window 6 on which can repose a layer 13 that substantially absorbs and re-emits radiation which passes through the window 6.

### CROSS-REFERENCE TO RELATED APPLICATIONS

This application claims the benefit of U.S. Provisional Patent Application No. 60/638,016 that was filed on Dec. 21, 2004, the disclosure of which is incorporated herein by reference.

### BACKGROUND OF THE INVENTION

1. Field of the Invention

The present invention relates to a portable, needle x-ray device for use in delivering high dose rates of x-ray radiation to a specified region of the body for treatment.

2. Background Art

Conventional medical x-ray sources used for radiation treatment are large, fixed position machines. Such machines operate in 150 kV to 20 MeV region depending on the desired depth of radiation treatment. Since present radiation therapy machines apply x-ray radiation to target regions internal to a patient from a source external to the target, substantial damage can be done to healthy tissue surrounding the area of treatment.

An alternative form of radiation therapy, called brachytherapy, involves implanting encapsulating radioisotopes in or near a tumor to be treated. While such use of radioisotopes may be effective in treating certain types of tumors, there is little ability to provide selective control of exposure (turn-on and turn-off) treatment radiation parameters. Handling and disposal of such radioisotopes involves hazards to both the individual handler and the environment.

Another invasive approach to radiation treatment is utilization of so-called miniaturized probe-type x-ray tubes, which are implantable into a patient's body for direct delivery of x-ray radiation (See e.g., U.S. Pat. Nos. 5,153,900; 5,428,658; and 5,442,678, the disclosures of which are incorporated here by reference.) Although direct irradiation of tumors looks promising, the known insertable miniaturized x-ray tubes have many limitations. The major disadvantages include: (a) difficulties with focusing and steering the electron beam to the target due to the collision of electrons en route to the target (such background x-rays could be as large as 40% of the primary dose rate at the needle tip)—also the tube must be shielded to avoid external electric and magnetic fields deflecting the beam to cause unwanted “leakage” lateral radiation that may impinge upon surrounding healthy tissue; (b) a necessity to cool the target to increase x-ray production efficiency; (c) impracticability of reducing the diameter of the miniaturized x-ray tube diameter below 4 mm (the requirement to decrease the diameter of the x-ray tube and the need to have a cooling system for the target are mutually exclusive); (d) significant limitations in changing shape of the emitted radiation, and (e) a high operating voltage of about 50-100 kV that is dangerous for the human body.

An improved implantable x-ray device was described in U.S. Pat. No. 6,580,940, the disclosure of which is also incorporated here by reference. In that device, an external x-ray source was used for delivering x-ray radiation into an implantable needle through a collimator. A pseudo-target positioned inside the implantable needle was used to produce treatment radiation.

Nevertheless, there remains a need for a relatively small, easily manipulated, controllable, low-energy, high dose rate insertable “x-ray scalpel” device that produces a narrow pencil-type x-ray beam and offers an x-ray source that can be positioned in close proximity to the area to be irradiated.

It would be desirable for the depth-dose distribution of x-ray radiation produced by such a device to be primarily localized in the volume of tissue to be treated while minimally affecting the surrounding healthy tissue. It would be also desirable to deliver much higher doses of radiation with considerably higher precision to the designated area of tissue in comparison to an implantable needle device with a secondary target.

More specifically, it would be desirable to deliver an x-ray beam of high intensity with precision to a limited volume of space to be treated. For example, if there is an area of, say, 1 mm³ that needs treatment, it would be desirable to not only focus a high energy x-ray beam to that area, but also to control beam parameters so that the beam is almost entirely absorbed in that 1 mm³ area to be treated.

An implantable high dose rate “x-ray scalpel” device operating at low x-ray energy will be suitable for many applications, such as radiation treatment of tumors and non-tumors disorders (e.g. nodular lesions, epilepsy, etc.).

### SUMMARY OF THE INVENTION

Thus, it is an object of the present invention to provide a portable, easily manipulated, high dose rate, low x-ray energy needle device.

Another object of the invention is to provide a portable, high dose rate, low-energy needle x-ray device having a collimator with incorporated conditioning optics (such conditioning optics can range from using an aperture to limit the x-ray beam to using capillary optics, a graded multilayer mirror or a highly oriented pyrolytic graphite ellipsoidal concentrator to create a focused or collimated beam).

It is a further an object of the invention to provide a portable, high dose rate, low energy needle x-ray device which is implantable into a patient for directly irradiating a desired volume of tissue.

Yet it is another object of the invention to provide a portable, high dose rate, low energy needle x-ray device for irradiating a specific region of the body according to a pre-calculated radiation fall-off profile inside the treated area in order to reduce tissue damage outside the desired irradiation region.

It is yet another object of the invention to provide a portable, high dose rate, low energy x-ray device with an applicator mounted thereupon for treating a desired surface region of the body.

It is a still further object of the invention to provide a portable, high dose rate, low-energy x-ray device and reference frame assembly (e.g. stereotactic system or robotic arm) for controllably positioning the implantable needle within a patient's body in order to irradiate the desired region.

A further object of the present invention is to allow adjustment in energy (e.g. by a tube and/or needle with a suitable metal coating on the exit window), flux intensity, and shape of the x-rays delivered to the tissue by utilizing an x-ray tube. Conditioning optics (e.g., multilayer optics, crystal x-ray concentrator, capillary optics, aperture, etc.) may be used to direct x-ray radiation to the implantable needle with a means for shaping x-ray radiation.

According to the present invention, there is provided an apparatus for radiation treatment by delivering x-ray radiation directly to a desired region of tissue, including tumors.

Briefly, the present invention includes an easily manipulated, portable, high dose rate apparatus having as an x-ray source, an x-ray tube of adjustable intensity, a collimator with one or more incorporated capillary lenses and an implantable needle. The x-ray tube is conjugated with the collimator which, in turn is conjugated with the implantable needle, which has an output window at its terminating end through which a treatment x-ray beam passes. The implantable needle may be fully or partially positioned into a patient to irradiate a desired region with x-rays.

The output window of the needle is made of an x-ray transparent material such as a plastic, a metal (such as beryllium) or a ceramic, such as boron carbide or boron nitride. By applying a metallic layer on the inside or outside (or both) of the output window, the shape and the energy of the x-ray beam produced can be changed, provided that the energy of the absorption edge (K-edge) of the deposited metal is lower than the energy of the x-ray beam passing through the output window. Thus, the needle x-ray device of the present invention allows adjustment in dose rate and shape of the x-rays delivered to an anatomical site of interest.

The needle x-ray device of the present invention avoids damaging the healthy tissue surrounding the area of radiation treatment. To achieve a desired radiation pattern over a desired region, while minimally irradiating other regions, the x-ray beam is emitted from a nominal position of the implantable needle located within or adjacent to the desired region to be irradiated. The disclosed invention can provide the required dose by irradiating any part of the desired region, either continuously or periodically, over extended periods of time.

### BRIEF DESCRIPTION OF THE DRAWINGS

FIG. 1 is a schematic representation of a system showing an x-ray source, an optical collimator and a needle;

FIG. 2 is a schematic representation of an x-ray beam focused on an exit window of the implantable needle;

FIGS. 3a and 3b are schematic representations of a system showing arrangements of two semilenses and a diaphragm;

FIG. 4 is a schematic representation of an optical focus shift for an x-ray beam passing through a needle;

FIG. 5 is a schematic representation of a focused x-ray beam;

FIG. 6 is a schematic representation of an x-ray beam focused on a thin metal plate detached from the output window of the implantable needle;

FIG. 7 is a schematic representation of an x-ray beam focused on a semi-transmitted metal plate detached from the output window of the implantable needle and positioned inside the tumor;

FIG. 8 is a graph which depicts dose variation with distance in a polymethylmethacrylate (PMMA) phantom;

FIG. 9 is a depiction of 2D intensity distribution of the produced quasi-parallel beam at the exit window of the needle x-ray device;

FIG. 10 is a graph of intensity versus energy for the x-ray radiation produced by the needle device with an exit window having a deposited Ti layer; and

FIG. 11 depicts an x-ray transmission spectrum for a 100 micron thick polyamide exit window.

### DETAILED DESCRIPTION OF THE PREFERRED EMBODIMENT(S)

1. The Apparatus

The invention includes an insertable needle-based x-ray system that is capable of administering an elevated dose rate. The system includes conditioning optics that is incorporated into the x-ray system in order to provide a high intensity x-ray beam. The x-ray system delivers radiation with a predetermined energy, intensity, and spatial distribution to or towards a selected area of the anatomy—for example, a tumor.

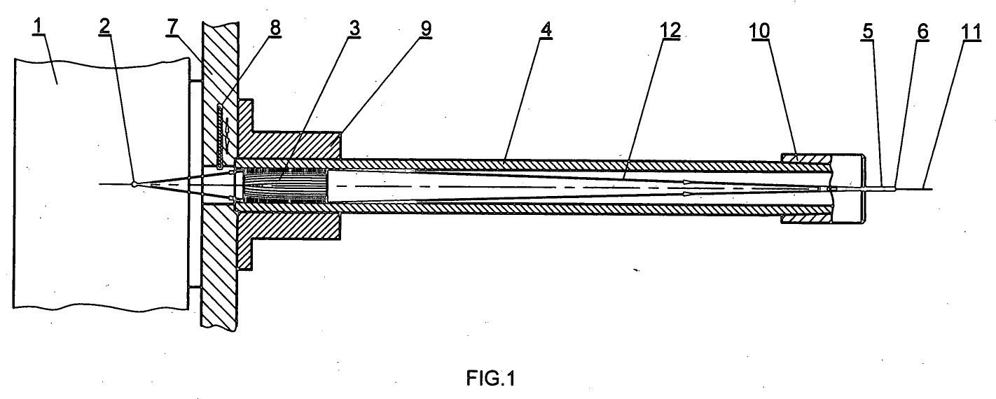

FIG. 1 shows one embodiment of an x-ray system containing an x-ray source 1 with a point focus 2, a capillary lens 3, an optical collimator 4 linked to the x-ray source 1 through a collimator holder 9, a needle holder 10 attached to the optical collimator 4, preferably through a Morse cone connection, or other connection means that provides consistent alignment and a secure, yet interchangeable interference fit and an implantable needle 5 with an output window 6 at its terminating end.

The x-ray system uses a focused x-ray beam 11 that is delivered through the implantable needle 5 that is optically conjugated with the focus 2 of the x-ray tube 1 through the optical collimator 4. Passage of the x-ray beam (on/off) may be controlled by a shutter 8 attached to the x-ray tube 1, preferably through a flange 7. If desired, the capillary lens 3 may focus the x-ray beam 11 on or in the vicinity of the output window 6 of the implantable needle 5. The output window 6 is substantially transparent to x-rays (i.e. for E=4.5 keV, more than 75% of incident x-rays pass through). A metal layer (e.g. titanium) can be applied (e.g., by a deposition process) onto a surface (preferably inside) of the output window 6 to modify the shape of the x-ray beam 11 passing through the output window 6. The implantable needle 5 has walls that are opaque to x-rays.

An x-ray tube that may be used as an x-ray source may have a point or a linear focus. For a linear focus, a take-off angle is chosen so that the projection of the linear focus on the plane perpendicular to the optical axis of the x-ray system is a point.

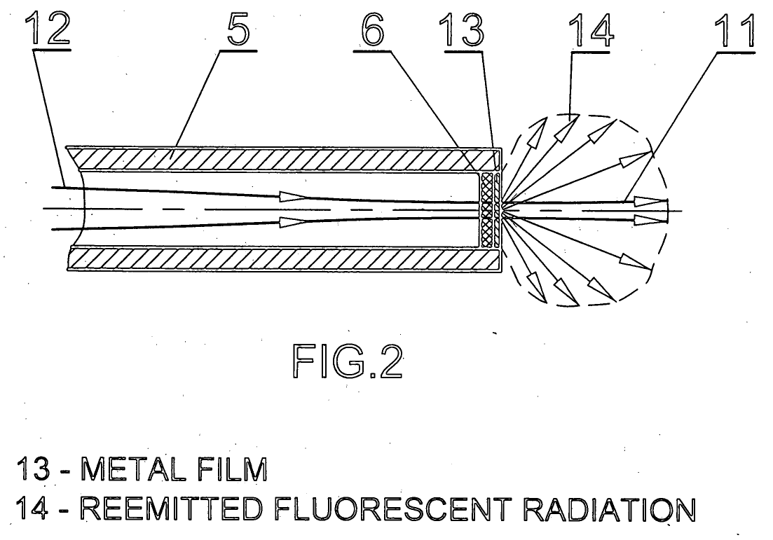

FIG. 2 shows a schematic representation of an x-ray beam 12 focused on the output window 6 of an implantable needle 5. The outer surface of the output window 6 can be coated with a layer 13. The x-ray radiation passing through the output widow 6 is a combination of a transmitted beam 12 and x-ray fluorescence 14 of the layer 13.

In one embodiment, the output window 6 is made of beryllium, carbon, boron carbide, boron nitride (or one or more other ceramics) or a plastic material (e.g. a polyamide) that is compatible with biological tissue. In another embodiment, the output window has a layer of a metal or metallic alloy for example, containing titanium applied to an external surface. In one embodiment, a thin film titanium layer has an absorption of x-rays that exceeds 90%. This allows the x-ray beam produced by the layer to be changed in its shape (space distribution) and in re-emitted energy. In one experiment as an example, the layer of titanium was from 2 microns to about 100 microns in thickness.

In a preferred embodiment, the collimator comprises a capillary lens. In one form, the capillary lens includes a plurality or band of capillaries having a complex curvature that is selected to produce the desired beam profile. They capture a divergent beam that is produced by the x-ray source and transform it into a parallel collimated (i.e. or quasi-parallel) or focused beam with a high intensity. Preferably, the ratio between the maximum diameter of capillary lens and the needle diameter does not exceed 4. For low energy x-ray radiation (below 8 keV), it is advantageous to fill the collimator with an inert gas such as helium (or a vacuum) in order to reduce the absorption of x-ray radiation inside the collimator.

In capillary optics-based collimation, x-rays incident on the interior of a narrow capillary (channel) at small angles (less than the critical angle for total internal reflection) are guided down the tubes. By assembling a number of hollow capillary tubes, a special arrangement can be formed. See, e.g., Kumakhov Mass., “Capillary X-Ray Optics—Introduction”, nuclear instruments and methods, B48, 283-9 (1990). See, also, Arkd'evVA et al., “New Components For X-Ray Optics”, Sov. Phys. USP 32, 271-6 (1989). Each of these publications is incorporated herein by reference.

In another preferred embodiment, the collimator comprises a bent, highly oriented pyrolytic graphite ellipsoidal concentrator. Suitable concentrators are available from the IFG Institute for Scientific Instruments (Berlin, Germany).

In yet another preferred embodiment, the collimator comprises graded multilayer mirrors mounted in a configuration selected from a Kirkpatrick-Baez scheme and an ellipsoid of rotation or a paraboloid of rotation.

Such assemblies can control x-ray beams, including collecting divergent radiation from a point source and transform them into one or more collimating or focusing beams.

Transformation efficiency of the capillary lens depends on a number of parameters including the capillary materials and configuration, the energy of incident x-ray radiation, point focus size, capture angle, and the radius of curvature of the capillaries.

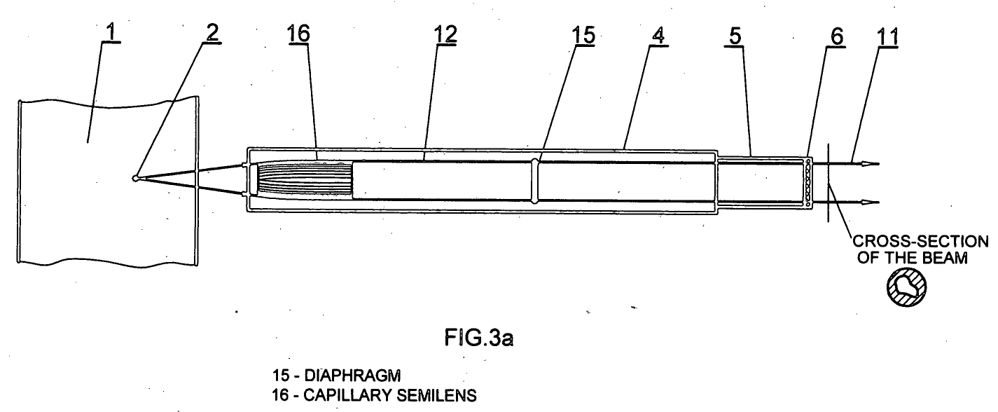

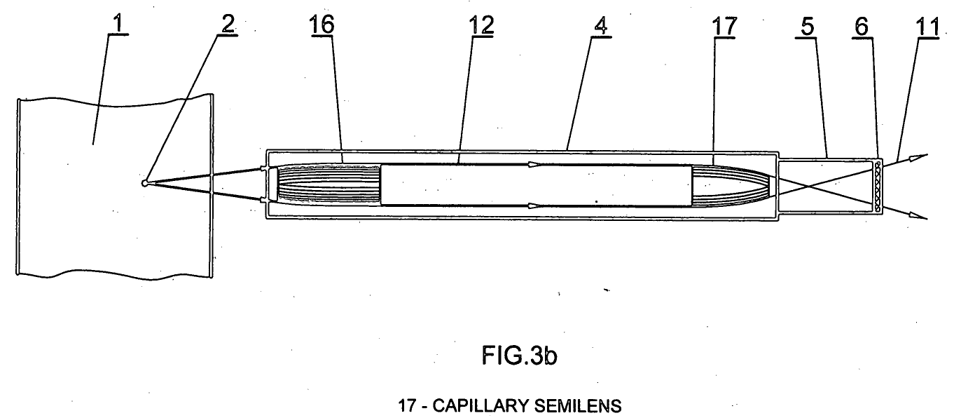

In one embodiment, the x-ray beam produced is focused on the output or exit window of the implantable needle. However, in other embodiments, the focus of the x-ray beam produced can be shifted along the axis of the needle. FIG. 3a shows an embodiment using an x-ray source 1 and a collimator 4 with one or more diaphragms 15 and a capillary semilens 16. The capillary semilens 16 transforms divergent radiation from the x-ray source 1 into a parallel beam 12. This parallel beam passes through the diaphragm 15 and the exit window 6. The one or more diaphragms 15 allows one to shape the beam 12 with precision.

In an alternate embodiment, it is also possible to use multiple semilenses 16 and 17 (FIG. 3b). In this approach a parallel x-ray beam 12 falls onto the second capillary semilens 17, which transforms it into a convergent (focused) beam.

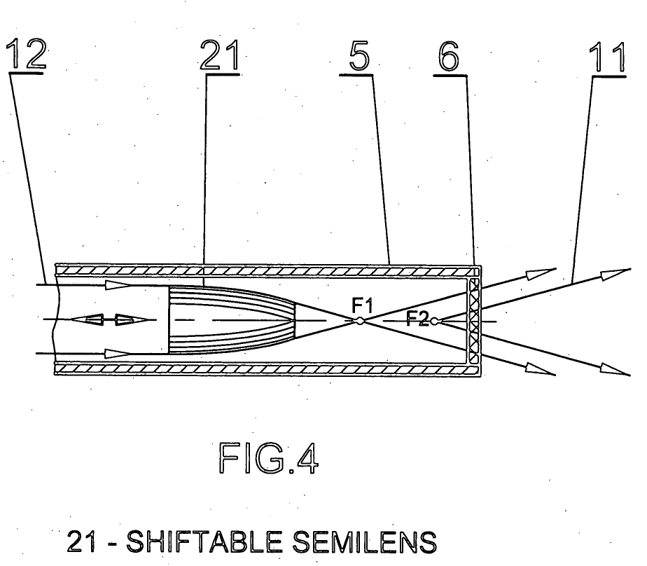

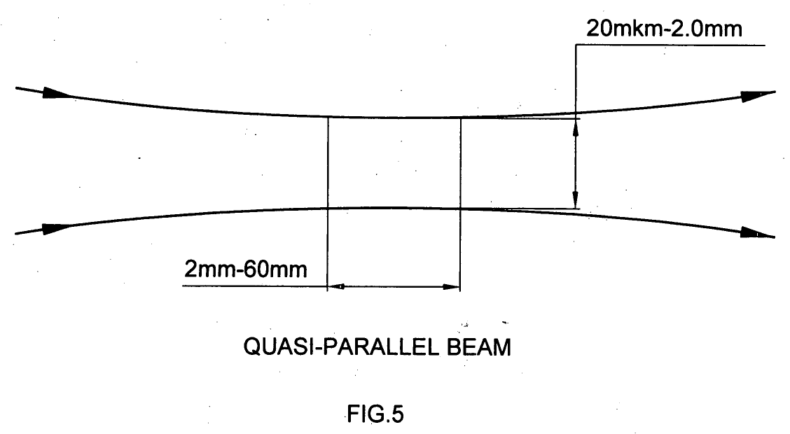

FIG. 4 schematically represents a shifting of an imaginary point optical focus (from position F₁ to position F₂) of the x-ray beam 12 with the shifting of the capillary lens 17 along the optical axis of the implantable needle 5. Angular spread of the x-ray beam 11 passing though the output window 6 depends on the position of the optical focus. In general, however, the “point” focus has dimensional attributes. Accordingly, the size of the focus can vary from 20 microns to 2 millimeters in diameter and the depth of the focus can vary from 2 mm to 60 mm (see FIG. 5) so that the surgeon has considerable, yet precise flexibility.

Thus, there is disclosed a needle x-ray device which is a source of a low energy, high dose rate, x-ray beam for treatment with a shape that can be changed from a parallel beam to a convergent beam or to a divergent beam. The device is designed to produce a radiation dose rate (e.g., up to 30-50 Gray/min) constrained within a beam with a diameter that can be changed from a fraction of a millimeter to 5 mm. The formed beam has a sharp drop-off curve for low energy x-rays.

As contemplated, the device promises to be useful for treating various anatomical sites of interest, e.g., highly localized disorders of the brain, including both tumor and non-tumor disorders.

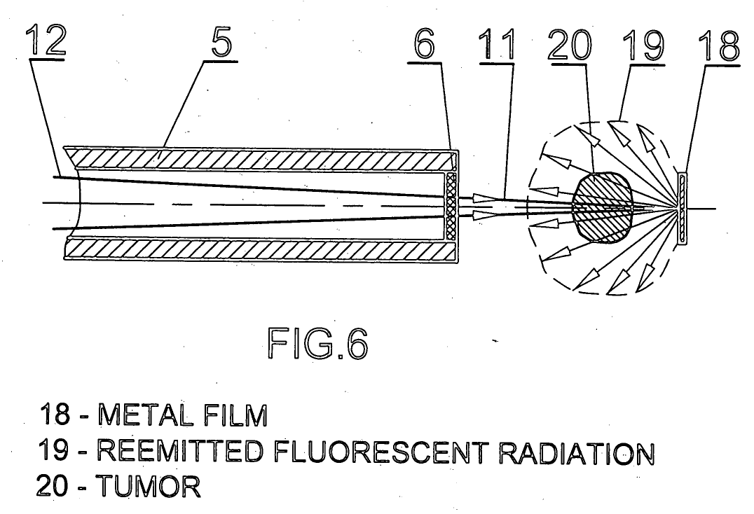

For example, a thin titanium plate (K absorption edge 4.9 keV), serving substantially as a shield and a secondary target can be positioned in front of a critical healthy organ 22 (FIG. 6) (such as the spinal cord) that needs to be protected and isolated from incident x-rays. A radiation beam with an energy of 5.4 keV (X-Ray tube with a Cr anode) can be used to excite the x-ray fluorescence of the titanium plate.

FIG. 6 depicts a still further alternate embodiment of the invention. In that embodiment, an x-ray beam passing through the output window 6 at the terminating end of the implantable needle 5, traverses a tumor 20, and is focused on a detached thin metal plate 18 or platelet (e.g. 100-200 microns thick)—its surface area is slightly larger than the beam cross-section. In this embodiment, the x-ray fluorescence 19 emitted by the thin metal (e.g. titanium) plate 18 irradiates the tumor 20. The thin plate 18 effectively serves as a detached x-ray source.

After the incident x-rays 11 pass through the tumor 20, they are remitted backwardly in the direction from which they came in a fan-like pattern 19, thereby isolating them from the healthy critical organ 22. The task of positioning the detached platelet 18 between the tumor 20 and the healthy organ 22 is a task that is accomplished following conventional medical/surgical procedures.

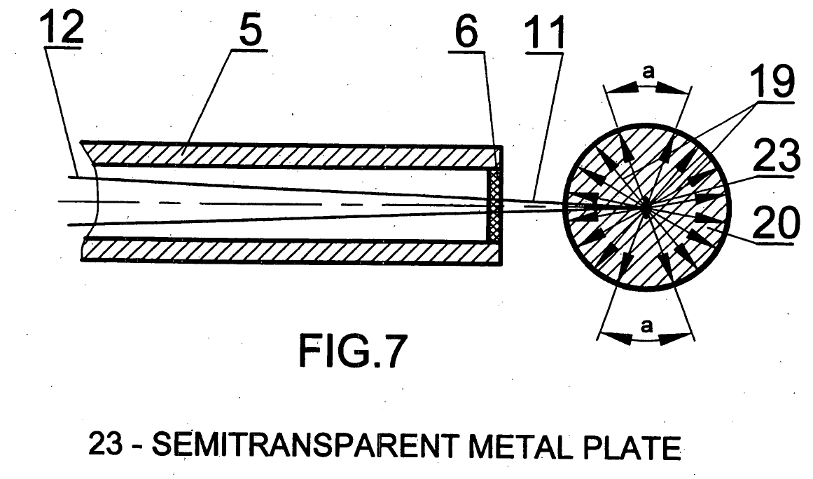

FIG. 7 depicts a still further alternate embodiment of the invention. In that embodiment, an x-ray beam passing through the output window 6 at the terminating end of the implantable needle 5, is focused on a detached, preferably less than 50 microns thick, semitransparent metal plate 23 placed inside the tumor. The surface area of this semitransparent plate is slightly larger than the cross-section of the incident x-ray beam. In this embodiment, the x-ray fluorescence 19 emitted by the thin plate 23 irradiates the tumor 20. The thin titanium plate 23 effectively serves as a detached x-ray source, emitting radiation uniformly in a sphere. A narrow zone is not irradiated (“x-ray fluorescence shadow”). The x-ray fluorescence shadow is about 10 degrees (angle alpha in FIG. 7). It can be eliminated by changing the position of plate 23 during treatment. The same principle (detached target) can be used for intraoperative radiation treatment after the tumor has been removed (e.g. for irradiating a lumpectomy cavity).

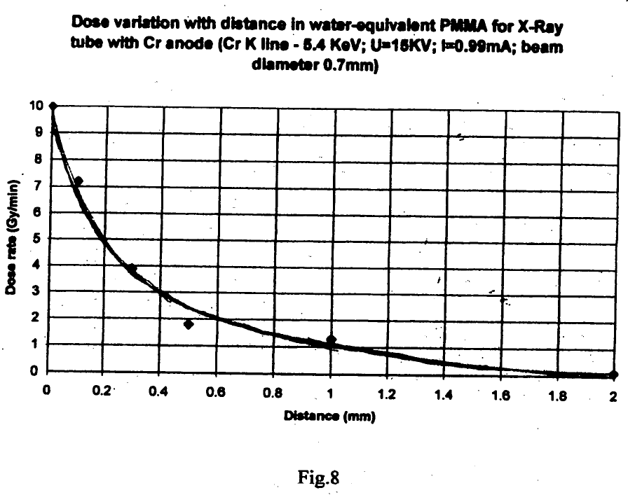

FIG. 8 is a graph that depicts dose rate variation with distance in PMMA phantom that simulates the optical density of tissues. An x-ray tube with a Cr anode (E=5.4 keV, U=15 kV, I=0.9 mA) was used as a primary x-ray source. The graph illustrates a characteristic fall-off curve that illustrates how the exit dose rate (10 Grays per minute) changes with distance (in millimeters) into the tumor. The dots in the FIG. 7 show experimental data obtained by measuring the dose of the produced x-ray radiation with thermo luminescent detectors (1×1×1 mm LiF crystals). A 6 meV Linac machine (Varian) was used for calibrating the detectors.

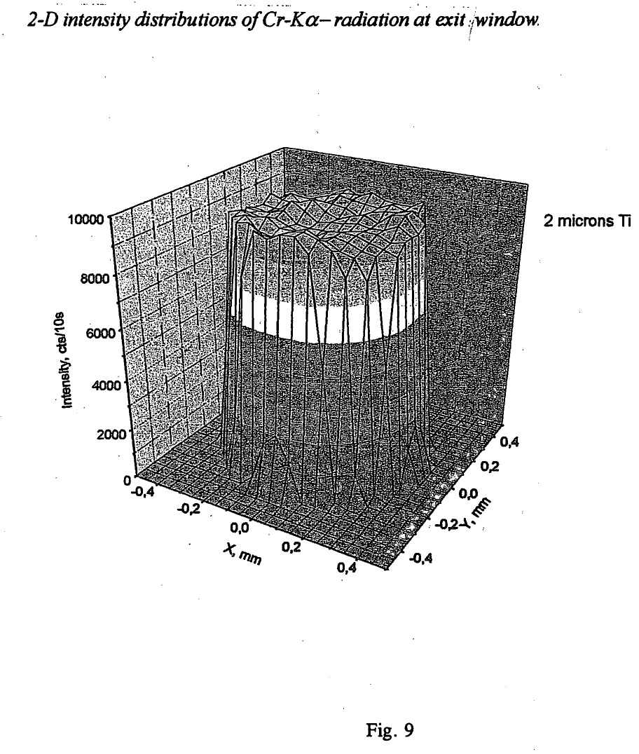

FIG. 9 shows a 3D intensity distribution of the produced quasi-parallel beam at the exit window of the needle x-ray device using an x-ray tube with a Cr anode as a primary x-ray source (E=5.4 keV). In this example, the diameter of the quasi-parallel x-ray beam was 0.7 mm. A multichannel silicon drift detector (SDD) with a 2.6 mm diameter window was used for x-ray beam intensity registration. The measurements were made by scanning a SDD detector with a 50 micron pinhole aperture across the x-ray beam produced.

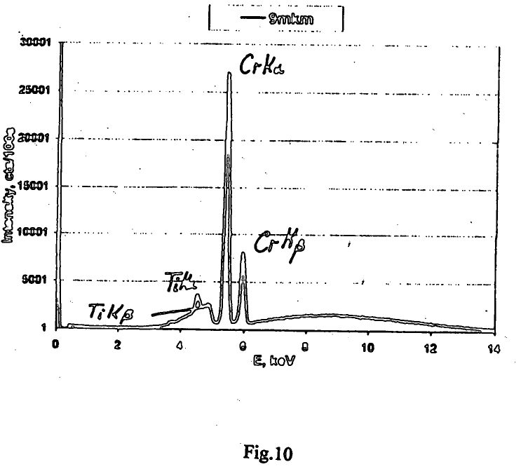

Turning now to FIG. 10, there is a graph of intensity versus energy for the x-ray radiation exiting the Ti-coated window of the needle device. The Ti coating was 9 microns thick. The detector was positioned at a 2 mm distance from the exit window. The spectrum in FIG. 9 contains Cr K_{a,} Cr K_{b,,} Ti Kₐ and Ti K_{b} lines. This indicates that the x-ray beam produced was a combination of a quasi-parallel x-ray beam generated by the x-ray tube with a Cr anode and a fan-type Ti x-ray florescence beam emitted by the Ti coating deposited on the exit window of the needle device.

For a 9 microns thick Ti coating, the measured transmission Iₚ=10%. Radiation produced by the x-ray tube with a Cr anode (E=5.4 keV) was effectively absorbed by the Ti coating (absorption edge E=4.97 keV). This results in about 35% efficiency of x-ray florescence I_{f} remitted by the Ti coating (X-ray data booklet, LBNL, Berkley, Calif., 2001, p. 1-28). Thus, it can be estimated that I_{f}=3 Iₚ (1).

As can be seen from the experimental curve (FIG. 10), I_{f}=0.14Iₚ (2). The experiments were carried out using a SDD detector having a 2.6 mm diameter sensitive element. Since the diameter of the quasi-parallel beam was 0.7 mm (smaller then the diameter of the sensitive element) the intensity Iₚ was fully measured by the detector. Radiation from the Ti coating was emitted into a sphere with a r=2 mm (i.e. distance from the exit window to the detector) and measured at by a SSD detector with a sensitive element having a surface area s=3.14 mm². This meant that only a small part of the I_{f} was measured by SSD detector. The measured part of the I_{f} can be calculated as follows: S^{d}/Sₛₚₕₑᵣₑ=0.04 (3). Thus, the measured intensity I_{f} was reduced 25 times. Any experimental curve containing I_{f} intensity was corrected using this coefficient. Taking into account (3), the experimental relation (2) can be changed to I_{f}=3.5Iₚ (4). Since the experimental data (4) is quite close to the estimation (1), it appears that the shape of the x-ray beam profile could be effectively changed by depositing a Ti coating on the exit window of the needle device.

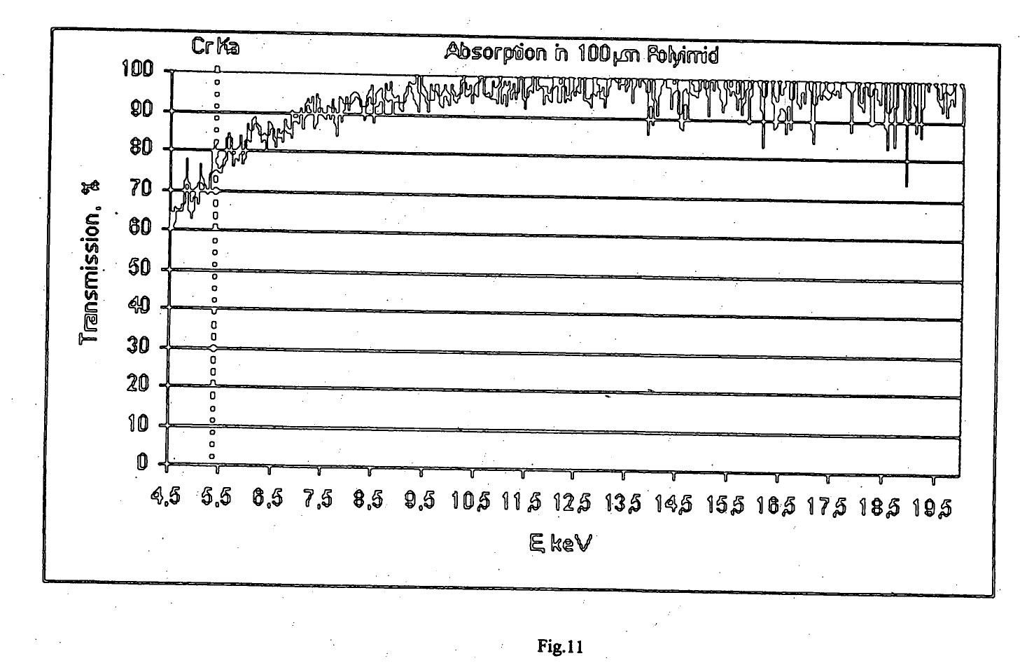

FIG. 11 is a graph showing x-ray transmission spectrum for a 100 micron thick polyamide exit window. This high endurance, tissue compatible and easy to clean material had a good transmission characteristic (more than 90%) at energies above 8 keV.

2. The Method

The methodology of developing and using the disclosed high intensity x-ray source involves:

(1) designing, building and testing conditioning optics;

(2) incorporating such conditioning optics into an optical collimator or into a needle;

(3) incorporating if desired one or more diaphragms into an optical collimator;

(4) conjugating a collimator with other components of the needle-based x-ray system;

(5) calculating the x-ray dose distribution produced outside the output window, preferably by using Monte-Carlo simulation;

(6) carrying out experiments to characterize the dose and dose rate distribution of radiation exiting the output window, and

(7) measuring the scattering of the x-ray beam produced in tissue-simulating materials.

In use, a primary x-ray beam is generated using, in one embodiment, an x-ray tube 1 that is positioned outside the insertable needle (5, FIG. 1). The primary x-ray beam 12 is guided into the hollow needle using an optical collimator 4. A transmitting output window 6, which can be coated with a film 13 (FIG. 2), is installed at the terminating end of the hollow needle.

When the needle is inserted into or near an anatomical site of interest, e.g., a tumor, the treatment beam irradiates the site through the output window 6 with high accuracy. When a metal film-coated window is used, the treatment radiation passed through the output window 6 is a combination of a transmitted beam and x-ray fluorescence of the film excited by the incident primary beam. This approach allows one to modify (e.g., to broaden) the radiation beam used for treatment.

The low-energy, high intensity treatment beam efficiently interacts with tumorous tissue since the relative biological effectiveness (RBE) of photons increases with decreasing photon energy. In addition, the low-energy radiation treatment has a significantly increased efficiency, compared to high-energy x-ray photons, when treating hypoxic (oxygen-deprived) central areas of solid tumors that are about 10% of tumor volume.

This design overcomes such limitations of miniaturized x-ray tubes as the necessity to insert a high voltage, high vacuum device into a human body and the inability to irradiate small regions (e.g. about 1×1×1 mm³) with high accuracy.

Using the disclosed system, beam intensity can be varied by controlling the power (intensity) of the primary x-ray beam and/or by use of a diaphragm.

Lower energy (3 keV-20 keV) radiation can be obtained by using x-ray tubes with different anodes and by selecting suitable film layers that repose on the output window of the needle. This overcomes the only single energy option presently available for sealed radioactive sources and limited energies of miniaturized x-ray tubes that are available to date. This broadens the range of energies that can be used for treatment.

3. Other Features

It is anticipated that in practice, the system can be configured to deliver an extended energy range (3 keV-20 keV), high dose rate x-ray system using the disclosed x-ray focusing/collimating optics.

The disclosed system has the capability of delivering treatments used to localized tumor and non-tumorous disorders.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. An x-ray device for radiation treatments using a focused or collimated x-ray beam, the device comprising:
a needle conjugated with an x-ray source through a collimator, the needle having an output window at its terminating end through which an x-ray beam passes for treating an anatomical site.

2. The x-ray device of claim 1, wherein said needle has one or more walls that are substantially opaque to x-rays.

3. The x-ray device of claim 1, wherein said output window is substantially transparent to x-rays.

4. The x-ray device of claim 1, wherein said output window comprises a material selected from the group consisting of beryllium, carbon, boron carbide, boron nitride, sapphire and a plastic that is compatible with biological tissue.

5. The x-ray device of claim 1, wherein said output window has a layer of a metal-containing material that reposes at least partially on the output window, the layer allowing the shape of the x-ray beam to be changed.

6. The x-ray device of claim 1, wherein the intensity of the x-ray beam can attenuate in proximity to the anatomical site up to two orders of magnitude at a distance of 1 to 10 mm from the output window of the implantable needle.

7. The x-ray device of claim 5, wherein the layer has a transparency to x-rays from 10% to 90%.

8. The x-ray device of claim 1, wherein the x-ray source comprises an x-ray tube.

9. The x-ray device of claim 8, wherein the x-ray tube has a point or a linear focus.

10. The x-ray device of claim 1, wherein the collimator comprises one or more capillary lenses.

11. The x-ray device of claim 10, wherein one or more of the capillary lenses comprise a plurality of bent capillaries of complex curvature which capture a divergent beam produced by the x-ray source and transform it into a parallel or focused beam of higher intensity.

12. The x-ray device of claim 11, wherein the ratio between the maximum diameter of the capillary lens and the needle diameter does not exceed 4.

13. The x-ray device of claim 1, wherein the collimator is filled with an inert gas or a vacuum in order to reduce the absorption of x-ray radiation inside the collimator.

14. The x-ray device of claim 1, wherein the collimator comprises a bent, pyrolytic graphite, ellipsoidal concentrator.

15. The x-ray device of claim 1, wherein the collimator comprises graded multi-layer mirrors mounted in a configuration selected from a Kirkpatrick-Baez scheme, and ellipsoid rotation, and a paraboloid rotation.

16. The x-ray device of claim 4, wherein the x-ray beam is focused on the output window of the implantable needle.

17. The x-ray device of claim 4, wherein the focus of the x-ray beam can be shifted along the axis of the needle.

18. The x-ray device of claim 1, wherein the collimator includes one or more diaphragms and a capillary semilens that transforms divergent radiation from the x-ray source into a parallel beam that passes through the diaphragm and an exit window of the needle.

19. The x-ray device of claim 1, wherein the collimator includes multiple semilenses so that a parallel beam that emerges from a first semilens falls onto a subsequent capillary semilens that transforms the x-ray beam into a convergent beam, thereby irradiating a larger area of the anatomical site to be treated when the focal point lies inside the needle.

20. The x-ray device of claim 19, where a subsequent capillary lens may be shifted along the optical axis of the needle so that an angular spread of the x-ray beam that passes through an output window of the needle is influenced by the position of the optical focus.

21. The x-ray device of claim 20, wherein the size of the focus can vary for treatment purposes from 20 microns to 2 millimeters in diameter and the depth of focus can be varied between 2 millimeters to 60 millimeters.

22. The x-ray device of claim 1, wherein the device produces a radiation dose rate of up to 30-50 Gray/min. constrained within a beam having a diameter of 20 microns to 5 mm.

23. The x-ray device of claim 1, wherein the area of the anatomical site to be treated is sized down to about 1×1×1 mm³.

24. The x-ray device of claim 4, wherein the energy of radiation passing through the output window is between 3 keV and 20 keV.

25. The x-ray device of claim 1, further including a detached metal platelet that is positioned between an anatomical site to be protected and an anatomical site to be treated, the platelet absorbing and re-emitting incident x-ray radiation.

26. The x-ray device of claim 1, further including a detached metal semitransmitting platelet that is positioned inside a tumor or a cavity after the tumor has been removed, the platelet absorbing and re-emitting incident x-ray radiation in a spherical distribution.

27. A method of using an x-ray device for radiation treatments, the method comprising the steps of:
(a) placing a platelet that is substantially opaque to x-ray radiation between a tumor and a healthy anatomical site to be shielded or placing a semitransparent metal platelet inside a cavity after the tumor has been removed;
(b) placing a needle so that an output window thereof lies in proximity to a tumor, the tumor lying between the platelet and the output window; and
(c) delivering low energy, high intensity x-rays to the output window of the needle toward the tumor.

28. The x-ray device of claim 11, wherein the focus of the x-ray beam produced by the x-ray source is positioned on an exit window of the implantable needle.

29. The x-ray device of claim 11, wherein the focus of the x-ray beam can be shifted along the axis of the needle.

30. The x-ray device of claim 13, wherein the inert gas is helium.

31. A method of using an x-ray device for radiation treatments, the method comprising the steps of:
placing a needle so that an output window thereof lies in proximity to or within a tumor; and
delivering a low-energy, high intensity x-ray to the output window of the needle.
